(19) United States
(12) Patent Application Publication
(10) Pub. No.: US 2013/0184753 A1
(43) Pub. Date: Jul. 18, 2013

(54) SYSTEM FOR GRIPPING SPINOUS PROCESSES, AND USES THEREOF

(71) Applicants: Glenn Keiper, Eugene, OR (US); Jerome Levieux, Geneve (CH)

(72) Inventors: Glenn Keiper, Eugene, OR (US); Jerome Levieux, Geneve (CH)

(73) Assignee: SPINEART SA, MEYRIN (CH)

(21) Appl. No.: 13/823,601

(22) PCT Filed: Sep. 16, 2011
(86) PCT No.: PCT/FR2011/052133

§ 371 Date: Mar. 14, 2013

(30) Foreign Application Priority Data
Sep. 17, 2010 (FR) 10 57472

(51) Int. Cl. A61B 17/70

(52) U.S. Cl. CPC A61B 17/7047
USPC 606/248

(57) ABSTRACT

A system for gripping spinous processes, includes a first plate (1) and a second plate (20), which are installed face to face, and a shaft (3), which is installed approximately perpendicularly with respect to the two plates and passes at least partially through one of them, the plates including an inner face and an outer face, a surface of the inner face of the plates is provided with raised roughening features (6), the first plate is movable relative to the second plate, the first plate is fixed in translation with respect to the shaft, and the shaft and the second plate form a non-return pawl mechanism including a series of notches that cooperate with the non-return pawl mechanism such that the movement of the plates toward each other is irreversible without external aid, whereby spinous processes can be effectively gripped between the two plates.

The present invention relates to a system for gripping spinous processes, and to uses thereof.

Diseases of the spine are treated in various ways depending on their severity and their particularities. One of these ways is by surgery. In particular, immobilization of two or more adjacent vertebrae is an established and effective technique. This blockage of two or more vertebral segments leads to two or more vertebrae becoming fused together.

There are several methods for fixing these vertebrae, the best known being the one that involves implanting pedicle screws and connecting these with longitudinal bars. Another method involves gripping the spinous processes of the affected vertebrae in order to block their relative movement. For this purpose, an interspinous fusion device is used.

Various systems of this type are available on the market, in particular the system sold by Medtronic under the name SPIRE Plate® or the system sold by Lanx under the name ASPEN®.

Although the SPIRE Plate system works well as regards gripping the spinous process, it is somewhat awkward to install, because the surgeon has to keep the two plates clamped and at the same time tighten a nut. This maneuver generally requires assistance, since it is difficult to perform with just two hands. Moreover, the SPIRE Plate system does not provide a zone in which the surgeon can place a bone graft intended to fuse the two spinous processes.

The ASPEN system provides a cylinder in which the surgeon can place the bone graft. However, this cylinder is made of titanium or of metal material and does not permit easy visualization of the graft during radiography checks. Moreover, like the SPIRE Plate® system, it is awkward to install. The principle is the same, and the surgeon therefore has to maintain the pressure on the spinous processes when tightening the blocking screw. Assistance is therefore needed with this device too, since the maneuver is easier with four hands.

US 2008/183211 describes an implant for spinous processes. The implant comprises a spacer and two plates engaged with the spacer, one of them fixed and one of them movable. The movable plate can be moved toward the fixed plate in order to stabilize the implant. The implant is fixed in place by blocking the movable plate, by means of tightening a screw against the rear face of the spacer. A screwing instrument is therefore needed to install the implant, and the use of the implant is therefore slow and awkward. In particular, the screwdriver with the appropriate bit must not be omitted.

It would be desirable to have novel systems available that are easier to fit in place and that do not require the tightening of a screw and, consequently, the use of a key or screwdriver in order to block the system. It would also be desirable to have novel systems available that do not require a spike or an analogous aid in order to block the system by spacing two parts of a component by insertion in between these two parts, especially because of the risk of losing this component.

It would also be desirable to allow the surgeon to fit a bone graft.

It would also be desirable to allow the bone graft to be monitored, in particular by radiography checks.

After lengthy research, the applicant has developed a novel device which affords complete satisfaction and is based on a self-locking system using a latching mechanism. Moreover, a chamber allows the surgeon to fit a bone graft.

It is for this reason that the present application relates to a system for gripping spinous processes, characterized in that it comprises a first plate and a second plate, which are installed face to face, and a shaft, which is optionally tubular and is installed approximately perpendicularly with respect to the two plates and passes at least partially through one of them, the plates comprising an inner face and an outer face, in that a surface of the inner face of the plates is provided with raised roughening features, in that the first plate is movable relative to the second plate, in that the first plate is fixed in translation with respect to the shaft, and in that the shaft and the second plate form a non-return pawl mechanism comprising a series of notches that cooperate with the non-return pawl mechanism such that the movement of the plates toward each other is irreversible without external aid, whereby spinous processes can be effectively gripped between the two plates.

A traditional example of a non-return pawl mechanism is the ratchet wheel, a device that allows a rotary mechanism to turn in only one direction. The circumference of such a wheel is provided with notches which, in the chosen direction, cause a pawl to lift in order to allow it to pass, but blocking the rotation in the other direction.

The first plate, fixed in translation with respect to the shaft, can be in one piece with the axle. However, under preferred conditions of use of the invention, the first plate and the shaft are two separate components. Since the spinous processes are of random shape, it is in fact desirable that a plate, preferably the first plate, is able to tilt at an angle with respect to the shaft.

Advantageously, when the first plate and the shaft are two separate components, one of the ends of the shaft comprises a circular hole arranged diametrically with respect to the axis of the shaft, for example in a ring shape.

The first plate can then comprise a cavity permitting passage of this end, and a means used as an axle for the circular hole in order to allow the shaft to move about a fixed point. This means can in particular be a screw, a peg, a pin, or a partially threaded pin. If its diameter is smaller than that of the radial circular opening, the first plate can move to a limited extent in a diametric direction of the shaft, about a fixed point situated in the axis of this means. Thus, the first plate is able to tilt at an angle with respect to the shaft, and the system for gripping spinous processes can adapt to spinous processes of varied shapes. The first plate and the second plate are not therefore systematically parallel.

The second plate can move in translation along the axis of the shaft in order to move toward the first plate and form a kind of vise. The second plate has a well that permits passage of the shaft and that allows the plates to be moved toward each other.

The shaft and the second plate form a non-return pawl mechanism, such that the movement of the plates toward each other is irreversible without external aid. This mechanism prevents the reverse movement of the second plate when the latter is moving toward the first plate.

Under preferred conditions of use of the invention, the shaft is a notched rod. The shaft can have any cross section, and this cross section is advantageously oval, particularly circular. One finds in particular a series of parallel grooves arranged on the periphery of the shaft or, in this latter case, a thread provided about the circumference.

In the case of a first plate that is fixed in translation with respect to the shaft, optionally in one piece with the axle, the gentle slope of a notch for blocking the movable plate will be arranged toward the free end of the shaft, while the steep slope thereof will be arranged toward the first plate.

Under other preferred conditions of use of the invention, in addition to the preceding ones, the well comprises one or more flexible blades, which in particular are fixed to the second plate at the proximal end and are free at the distal end. These flexible blades are preferably arranged approximately parallel to the axis of the shaft. Advantageously, the inner surface of said one or more flexible blades comprises a protrusion such as a sharp bead or a ridge or the like, preferably provided at the distal end or close to the distal end. The cooperation between the notches of the shaft and the protrusions reinforces the desired detent effect.

Under yet other preferred conditions of use of the invention, in addition to the preceding ones, the shaft is provided with one or more recesses for receiving a bone graft. This hole can in particular be a multiplicity of holes arranged close together in a honeycomb formation, for example. These recesses have a depth representing part of the cross section of the shaft (the cross section being a cut made through the shaft perpendicularly with respect to its axis). The recess can represent, for example, 10 to 90% of the cross section, preferably 15 to 80% of the cross section, especially 20 to 75% of the cross section, particularly 25 to 70% of the cross section, very particularly 30 to 65% of the cross section.

These recesses also have a certain length (in the direction of the axis of the shaft). This is the length of the recess, if there is only one of them, or the length of the whole of the recessed zone when there are several recesses (for example in the case of recesses arranged close together in a honeycomb formation). The length of these recesses will be from 1 to 20 mm, preferably 2 to 18 mm, especially 3 to 15 mm, particularly 4 to 12 mm, very particularly 5 to 10 mm.

A system for guiding the movement of the second plate along the shaft is advantageously provided. For this purpose, the shaft can be provided with a longitudinal channel that cooperates with a protuberance formed toward the inside of the well, for example in the area of the plate.

The shaft is advantageously made of polyether ether ketone (PEEK) or of another material having the same mechanical and radiotransparent properties, so as to permit visualization of the graft during radiography checks. However, it can also be made of other implantable polymer materials or metals, for example titanium, alloyed or pure.

The external diameter of the shaft will be, for example, 2 to 18, preferably 4 to 16, especially 5 to 15, very particularly 6 to 14 mm. For the shaft to serve also as an interspinous wedge (spacer), the external diameter of the shaft will be, for example, 5 to 20, preferably 6 to 20, especially 6 to 18, very particularly 8 to 18 mm.

The length of the shaft will be, for example, 20 to 50, preferably 25 to 45, especially 25 to 40, very particularly 30 to 35 mm.

The length of the plates will be, for example, 1.5 to 7, preferably 2 to 6, especially 2 to 5, very particularly 3 to 4 cm.

The maximum width of the plates will be, for example, 3 to 16, preferably 4 to 12, in particular 5 to 10, very particularly 6 to 8 mm.

The thickness of the plates will be, for example, 1 to 8, preferably 1.5 to 6, especially 1.5 to 4, very particularly 2 to 3 mm.

The length of the well excluding the plate (hence generally approximately the length of the blades) will be, for example, 2 to 14, preferably 3 to 12, especially 4 to 10, very particularly 6 to 8 mm.

The number of the blades will advantageously be 1 to 20, preferably 4 to 16, especially 6 to 12, very particularly 8 to 10.

The number of the grooves will advantageously be 4 to 35, preferably 6 to 30, especially 8 to 25, very particularly 10 to 20. The spacing between two grooves/notches (hollow to hollow or crest to crest) can range from 0.2 to 2.5 mm, preferably from 0.4 to 2 mm, especially from 0.6 to 1.5 mm.

The systems for gripping spinous processes, and forming the subject matter of the present invention, have very advantageous properties and qualities. They make it possible to easily block the relative movement of the vertebrae concerned. The elastic blades ensure the irreversibility of the movement by locking the position of the plates and maintaining the compression on the bone.

An important advantage of this system is the ease of its installation, and also its stability over time, obtained by virtue of the non-return pawl mechanism. It is fitted in place by a single tightening maneuver, with just one hand being needed. Moreover, there is no risk of a blocking screw coming loose over time, which is important because this type of device is designed to be implanted for a lifetime.

These qualities are illustrated hereinafter. They justify the use of the bar assemblies described below in the stabilization of the vertebral column.

It is for this reason that the present application also relates to a method for stabilizing the vertebral column, in which systems for gripping spinous processes, as described above, are installed on adjacent vertebrae, and said adjacent vertebrae are blocked. Preferably, a bone graft is also installed in the one or more recesses of the shaft.

The above devices being for surgical use, the present application also relates to said devices in sterile form, especially packed in a packaging that preserves their sterility.

The preferred conditions of use of the systems for gripping spinous processes, as described above, apply also to the other subjects of the invention set out above, especially to the procedures and methods using them and for their manufacture.

The invention will be better understood by reference to the attached drawings, in which

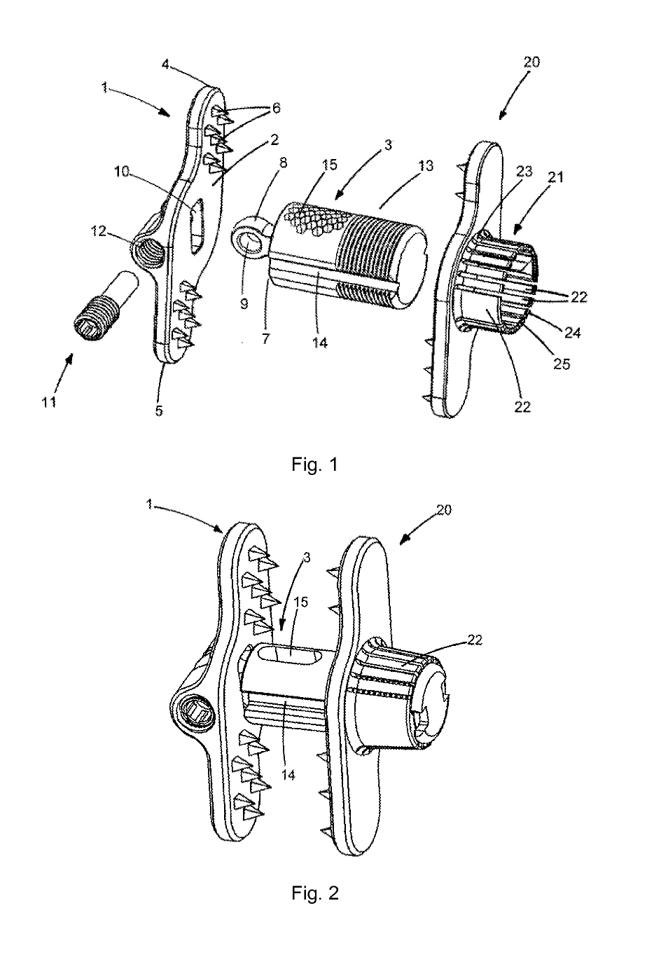

FIG. 1 shows a perspective view of a system for gripping spinous processes according to the invention, the components of which system are disassembled.

FIG. 2 shows such a system in a functional position.

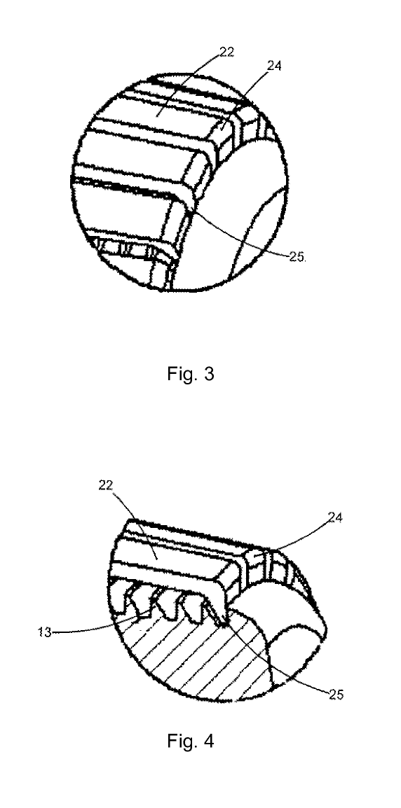

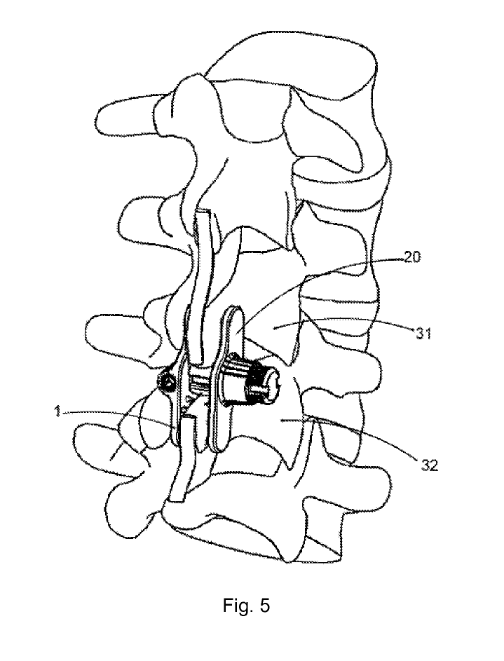

FIG. 3 shows an enlarged detail illustrating the pawl system, as does FIG. 4, which is a cross-sectional view thereof.

FIG. 5 shows a system according to the invention mounted on adjacent spinous processes.

In FIG. 1, a first plate 1 of elongate shape is seen on the left. The inner face 2 of this plate 1 is directed toward the shaft 3 and is provided with raised roughening features 6 at its first end 4 and its second end 5. These roughening features 6 are able to embed themselves in spinous processes, as will be seen below with reference to FIG. 5. The shaft 3 is generally cylindrical and solid. It comprises, at its first end 7, a circular opening which is arranged diametrically with respect to the axis of the shaft and which is a ring 8 comprising a hole 9. This ring 8 can engage in a cavity 10 provided in the first plate. This cavity 10 has a size that permits the pivoting of the shaft (from the top downward, and vice versa, in this figure). To secure the shaft 3 to the first plate 1, a threaded pin 11 is provided which is screwed into the threaded channel 12, provided in a hump formed on the first plate 1, in order to pass through the hole 9 of the ring 8 and block the translational movements of the shaft 3 with respect to the first plate 1, while at the same time allowing it to pivot.

This shaft 3 comprises, at its end opposite the ring 8, a series of twenty circumferential and mutually parallel grooves 13 constituting a latching mechanism. The system also comprises a second plate 20 with a structure comparable to the first plate as regards the plate itself. However, the center of the plate is provided with a well 21 formed by a set of blades 22 which are fixed via their base 23 to the plate 20 and of which the top 24 is free. Thus, these blades 22 are flexible. Toward the end 24 of the blades 22, each blade is provided with a sharp ridge 25 directed toward the inside of the well. These ridges 25 can cooperate with the grooves 13 of the shaft 3 by snap-fitting into said grooves 13 in order to render the movement of the first plate and second plate toward each other irreversible.

In order to prevent rotation of the second plate 20 relative to the shaft 3, two elongate and V-shaped channels 14 are provided diametrically opposite each other within the thickness of the wall of the shaft 3 and cooperate with protuberances of complementary shape (not shown) provided on the inside of the well, in the area of the plate. When the second plate has been pushed toward the first plate, which can be done with the aid of forceps, the flexible blades spread apart upon passage from one groove to another, so as to then move toward each other after passage of the protrusion and block themselves in the hollow of the following groove. As the shaft 3 is generally cylindrical and solid, it has no longitudinal slit separating the shaft into two parts in such a way that these two parts can be moved toward each other. Thus, the retaining force produced by the ridges 25 cooperating with the grooves 13 of the shaft 3 is constant from one end to the other of the grooves 13, whereas, if the shaft 3 had a longitudinal slit, the retaining force would be less at the opening of the slit remote from the area where the two parts join.

Recesses 15 are provided in the shaft 3 in order to allow bone marrow grafts to be fitted. These recesses 15 are honeycomb-shaped. In a cross section perpendicular to the axis, they represent, depending on the recess considered, a depth of 30 to 80% of the cross section of the shaft.

In FIG. 2, the system is assembled with a view to being used to grip spinous processes.

In this figure, the shaft 3 has a single recess 15 of oblong shape. It represents approximately 60% of the cross section of the shaft (cut made perpendicularly with respect to the axis of the shaft). The channels 14 have a U-shaped cross section instead of a V-shaped one. The ring 8 has been introduced into the cavity 10, then secured to the first plate with the aid of the partially threaded pin 11. This is provided with a hexagonal socket to permit screwing by an Allen key. The two plates 1, 20 are shown in parallel, but the first plate 1, on the left-hand side, is capable of pivoting, for example in order to better adapt to spinous processes of different thicknesses and random shapes.

In FIG. 3, which shows an enlarged detail from FIG. 2, the distal end 24 of the blades 22, and the ridges 25 thereof, can be better seen.

These parts are seen even better in FIG. 4, which shows the mutually parallel grooves 13 constituting a latching mechanism of sawtooth-shaped cross section. The ridges 25 at the end 24 of the blades 22 can also be better seen here.

In FIG. 5, two adjacent vertebrae 31 and 32 have been blocked by using a system according to the invention, by virtue of the spinous processes being gripped in the manner of a vise between the first plate 1 and the second plate 20.

In this model, the shaft 3 has been made from PEEK, while the three other components are made of titanium.

The diameter of the shaft 3 is approximately 12 mm and its total length, including the ring, is approximately 32 mm. The length of the plates is approximately 3 cm and their maximum width is approximately 1 cm. The thickness of the plates is approximately 2 mm and the length of the well, excluding the plate, is approximately 8 mm.

The relatively large diameter of the shaft 3 permits good cooperation between ridges 25 and grooves 13, since the blocking can take place about the entire circumference, or almost the entire circumference, of the shaft and, in addition, the shaft 3 can serve as a spacer between the spinous processes.

The following 2 systems of different size and nature have also been produced on the same principle:

| | 2 | | 3 | |
|---|---|---|---|---|
| Material used for the shaft | PEEK | | T40 | |
| Material used for the first plate and | Ta6v | | Ta6v | |
| the second plate | | | | |
| Material used for the pin | Ta6v | | Ta6v | |
| Height of the plates | 2.5 | cm | 3 | cm |
| Maximum width of the plates | 12 | mm | 8 | mm |
| Thickness of the plates | 2.5 | mm | 3 | mm |
| Length of the well excluding plate | 8 | mm | 12 | mm |
| Total length of the shaft | 25 | mm | 42 | mm |

1. A system for gripping spinous processes, characterized in that it comprises a first plate (1) and a second plate (20), which are installed face to face, and a shaft (3), which is installed approximately perpendicularly with respect to the two plates (1, 20) and passes at least partially through one of them, the plates (1, 20) comprising an inner face and an outer face, in that a surface of the inner face of the plates (1, 20) is provided with raised roughening features (6), in that the first plate (1) is movable relative to the second plate (20), in that the first plate (1) is fixed in translation with respect to the shaft (3), and in that the shaft (3) and the second plate (20) form a non-return pawl mechanism comprising a series of notches that cooperate with the non-return pawl mechanism such that the movement of the plates (1, 20) toward each other is irreversible without external aid, whereby spinous processes can be effectively gripped between the two plates (1, 20).

2. The system for gripping spinous processes as claimed in claim 1, characterized in that the external diameter of the shaft is 2 to 18 mm, preferably 4 to 18 mm, particularly 4 to 16 mm.

3. The system for gripping spinous processes as claimed in claim 1, characterized in that the external diameter of the shaft is 5 to 20 mm.

4. The system for gripping spinous processes as claimed in claim 1, characterized in that the first plate and the shaft (3) are two separate components.

5. The system for gripping spinous processes as claimed in claim 1, characterized in that one of the ends of the shaft (3) comprises a circular hole (9) arranged diametrically with respect to the axis of the shaft (3).

6. The system for gripping spinous processes as claimed in claim 5, characterized in that the first plate comprises a cavity (10), for passage of the end of the shaft (3), and a means (11) used as an axle for the circular hole (9) in order to allow the shaft (3) to move about a fixed point.

7. The system for gripping spinous processes as claimed in claim 1, characterized in that the second plate (20) has a well (21) permitting passage of the shaft (3) and allowing the plates (1, 20) to be moved toward each other.

8. The system for gripping spinous processes as claimed in claim 1, characterized in that the shaft is a notched rod.

9. The system for gripping spinous processes as claimed in claim 1, characterized in that a series of parallel grooves (13) is arranged on the periphery of the shaft (3).

10. The system for gripping spinous processes as claimed in claim 7, characterized in that the well (21) comprises one or more flexible blades (22).

11. The system for gripping spinous processes as claimed in claim 1, characterized in that the flexible blades (22) are fixed at the proximal end and free at the distal end.

12. The system for gripping spinous processes as claimed in claim 10, characterized in that the flexible blades (22) have a protrusion (25) on their inner surface.

13. The system for gripping spinous processes as claimed in claim 7, characterized in that the shaft (3) is provided with one or more recesses (15) for receiving a bone graft, the recesses for the graft being formed on the shaft 3.

14. The system for gripping spinous processes as claimed in claim 1, characterized in that the shaft (3) is provided with a longitudinal channel (14) cooperating with a protuberance provided toward the inside of the well.

15. The system for gripping spinous processes as claimed in claim 8, characterized in that the well (21) comprises one or more flexible blades (22).

16. The system for gripping spinous processes as claimed in claim 9, characterized in that the well (21) comprises one or more flexible blades (22).

17. The system for gripping spinous processes as claimed in claim 11, characterized in that the flexible blades (22) have a protrusion (25) on their inner surface.

18. The system for gripping spinous processes as claimed in claim 8, characterized in that the shaft (3) is provided with one or more recesses (15) for receiving a bone graft, the recesses for the graft being formed on the shaft 3.
